(19) United States
(12) Patent Application Publication
(10) Pub. No.: US 2026/0174423 A1
(43) Pub. Date: Jun. 25, 2026

(54) Tensionable Knotless Anchors and Methods of Tissue Repair

(71) Applicant: Arthrex, Inc., Naples, FL (US)

(72) Inventor: Jason A. VALENTIN, Fort Myers, FL (US)

(73) Assignee: Arthrex, Inc., Naples, FL (US)

(21) Appl. No.: 19/398,791

(22) Filed: Nov. 24, 2025

Related U.S. Application Data
(60) Provisional application No. 63/736,887, filed on Dec. 20, 2024.

(51) Int. Cl. A61B 17/04; A61B 17/06

(52) U.S. Cl. CPC A61B 17/0401; A61B 17/06166; A61B 2017/0414; A61B 2017/0618

(57) ABSTRACT

Tensionable, knotless, self-locking surgical constructs and methods for surgical repairs are disclosed. A surgical construct may be in the form of a soft suture anchor with a braid that may have tapered areas on one or both sides of the braid. The surgical construct may include one or more shuttle/pull devices to aid in formation of splices and tensionable, knotless loops.

### CROSS-REFERENCE TO RELATED APPLICATIONS

This application claims priority to US Provisional Application No. 63/736,887 filed Dec. 20, 2024, the disclosure of which is hereby incorporated by reference in its entirety herein.

### BACKGROUND

The disclosure relates to the field of surgery and, more specifically, to knotless anchor constructs and associated methods of tissue repairs.

### SUMMARY

Surgical systems, assemblies, and methods for fixation of soft tissue are disclosed.

A surgical construct may create a knotless, self-locking, reinforced repair. A surgical construct may be made completely of suture to achieve fixation in bone without a separate anchoring body and with increased fixation. The design enables fixation by deployment into bone with a suture tail remaining outside the bone for tensioning and/or alternative usage. The surgical construct may be employed in knotless fixation of first tissue to second tissue, for example, fixation of soft tissue to bone.

Methods of tissue repairs are also disclosed. In some implementations, a knotless surgical construct may provide tissue fixation without any knot formation, by providing an all-suture soft anchor that does not require a separate anchoring body or similar structure, and which allows increased tissue fixation.

These and other features and advantages of this disclosure will become apparent and will be understood from the following detailed description of the various aspects of the disclosure taken in conjunction with the accompanying drawings.

### BRIEF DESCRIPTION OF THE DRAWINGS

FIG. 1 illustrates an exemplary method of assembling a surgical construct at an initial stage.

FIG. 2 illustrates the surgical construct of FIG. 1 at a subsequent stage.

FIG. 3 illustrates the surgical construct of FIG. 2 at a subsequent stage.

FIG. 4 illustrates another view of the surgical construct of FIG. 3.

FIG. 5 illustrates a schematic repair with the surgical construct of FIG. 4.

### DETAILED DESCRIPTION

A surgical construct may create a knotless, reinforced, self-locking repair.

A tissue repair system may include a tensionable, knotless, self-locking surgical construct with a fixation device in the form of a tapered flexible strand (tapered flexible braid) with different widths and/or diameters on at least one end of the strand, and a shuttle/pull device. A tissue repair system may be a soft tissue repair system.

In some implementations, a soft suture anchor may include a suture provided with one or more tapered areas on at least one end of the suture. The suture may be a braid. The suture may be a suture tape. In some implementations, a soft suture anchor may include a suture provided with one or more tapered areas on each end of the suture. The suture may be a braid. The suture may be a suture tape. A tapered area on one end of the suture houses a knotless mechanism and acts as anchoring body. A tapered area on another end of the suture (the opposite end) can be modified to complement auxiliary instrumentation of implants.

The surgical construct may secure tissue (arthroscopic or endoscopic) with a flexible strand that does not require the surgeon to tie a knot. The construct pulls soft tissue together. The splices in the flexible strand increase the pull-out resistance of the construct and improve tissue cut-through resistance (by reducing the “cheese wire” effect) which is important in many clinical applications. The surgical construct may be employed in knotless fixation of first tissue to second tissue, for example, fixation of soft tissue to bone.

Methods of knotless tissue repairs are also disclosed. In some implementations, a surgical construct may provide fixation of a first tissue to a second tissue, for example, knotless fixation of soft tissue (ligament, tendon, graft, etc.) to bone without any knot formation and in a simple and fast manner.

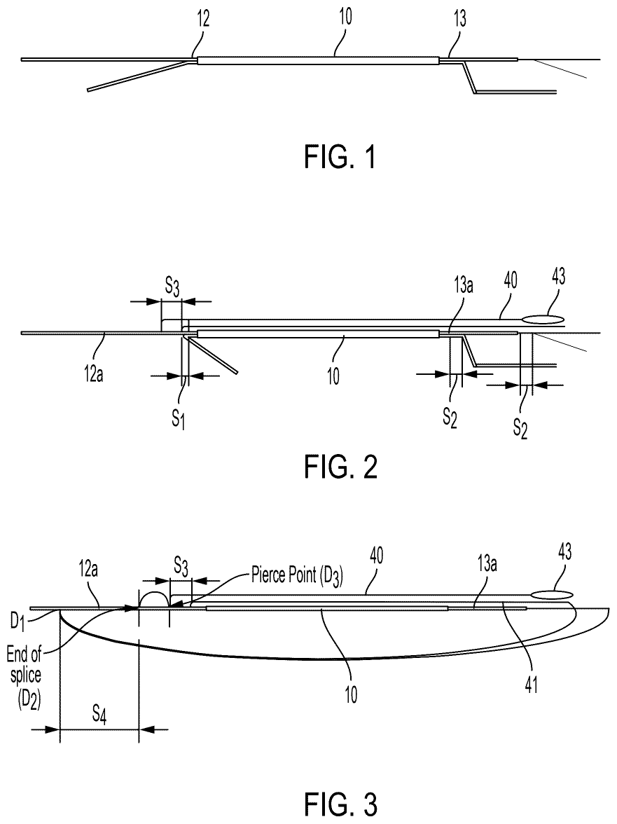

Referring now to the drawings, where like elements are designated by like reference numerals, FIGS. 1-4 illustrate exemplary steps of assembling surgical construct 100 including fixation device 10 (braid 10) with exemplary knotless mechanism 199 and shuttle/pull device 40. Mechanism 199 may be a self-locking mechanism. Mechanism 199 may be a tensionable mechanism. FIG. 5 illustrates an exemplary soft tissue repair 101 with surgical construct 100.

Surgical construct 100 may be an implantable device, a surgical assembly, a surgical system, a suture anchor, a tensionable, knotless construct, a tensionable, knotless, self-locking, surgical anchor, or a single suture knotless FiberTak® fixation device.

As detailed below, surgical construct 100 is an implantable device made completely of suture or sutures to achieve fixation in bone without a separate anchoring body. The design enables for fixation by deployment into bone with a suture tail remaining outside the bone for tensioning and alternative usage.

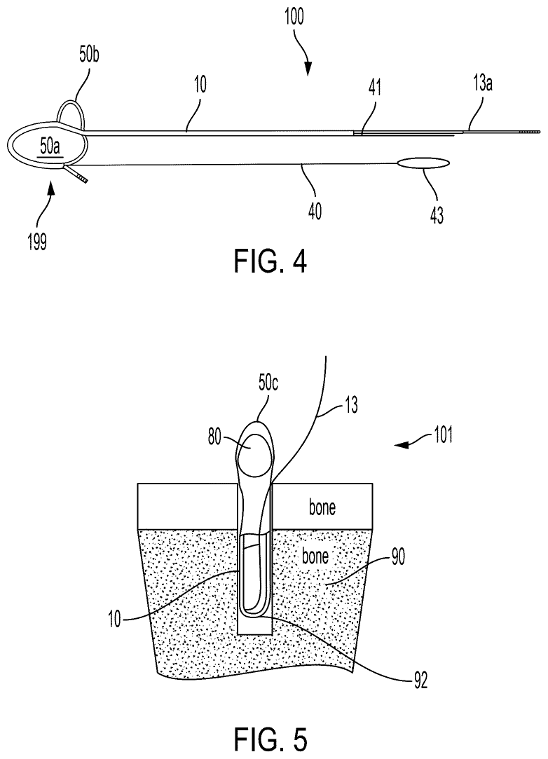

Surgical construct 100 of FIG. 4 may include fixation device 10 in the form of flexible coupler 10 provided with two ends 12, 13. Flexible coupler 10 may be a braid, a suture, a suture tape, a round-flat-round suture, or a repair suture. In some implementations, one end may be split into two tails (bifurcated), while the other end may be split into two tails (bifurcated) and then may be split again into two tails (again bifurcated).

Although the implementations below will be described with reference to flexible coupler 10 provided with two tapered ends (for an easier conversion of the knotless mechanism), it must be understood that the disclosure is not limited to these exemplary-only implementations and also contemplates implementations and embodiments where flexible coupler 10 is provided with only one tapered end. In certain implementations, the tissue fixation methods detailed below may be conducted with a flexible coupler having no tapered ends.

In some implementations, one of the two ends (for example, end 12) may be a tapered end with a downsized suture having at least a first constant diameter which is smaller than the diameter of the braid. The other end (for example, end 13) may be a tapered end with two or more regions of downsized suture having at least second and third constant diameters which are smaller than the diameter of the braid. The second and third diameters may be different from each other to allow for a tapered suture that makes it easier to convert the knotless mechanism, as detailed below.

In some implementations, braid 10 may be formed by cutting a braid off the spool and splitting the braid into a specific number of downsized sutures, as shown in FIG. 1. Splitting the braid into downsized sutures may be conducted in any manner and for any number of times to form one or more tapered ends and help with the formation of the final construct.

In some implementations, one end of the braid 10 (for example, the left end 12 in FIG. 1) may be bifurcated so that one bifurcated end (limb) is passed/spliced through the other bifurcated end (limb) to form a tail merge splice S1 (FIG. 2) within the other bifurcated limb and a tapered end 12a. The other end of the braid 10 (for example, the right end 13 in FIG. 1) may be bifurcated and then quadfurcated so that one bifurcated end (limb) is passed/spliced through the other bifurcated end (limb), and one quadfurcated end (limb) is passed/spliced through the other quadfurcated end (limb) to form two tail merges splices S2 (FIG. 2) within the right end 13 and a double-tapered end 13a. In this manner, once all remaining non-spliced tails are cut, the resulting structure is the tapered flexible coupler 10 of FIG. 3 with three tail merges splices S1, S2 provided on tapered ends 12a, 13a of braid 10.

In some implementations, flexible coupler 10 may be provided as a single strand of #5 or #2 machine taper suture. Flexible coupler 10 may be braided to a round suture with various diameters along its length. The diameter differentiation and furcation of suture may occur within the manufacturing process (e.g., braiding, weaving, sewing, stitching, etc.). In some implementations, flexible coupler 10 may be provided as a one-piece machine taper construct in the form of a round single suture strand of #5 or #2 machine taper suture. The flexible coupler 10 acts as a soft anchor.

The flexible coupler 10 may be manufactured from any flexible material, for example, multifilament, braided, knitted, woven suture, or including fibers of ultrahigh molecular weight polyethylene (UHMWPE) or the FiberWire® suture (disclosed in US 6,716,234, the disclosure of which is hereby incorporated by reference in its entirety herein). Surgical constructs may be used with any type of flexible material or suture known in the art. The flexible coupler 10 may include elastic material. The flexible coupler 10 may consist essentially of elastic tapered suture.

FIG. 2 also illustrates a shuttle/pull device 40 extending through and attached to the flexible coupler 10. Shuttle/pull device 40 (suture passing instrument 40; suture passer 40; shuttle link 40; FiberLink™ 40; nitinol loop 40) is provided attached to flexible coupler 10, by being spliced, for example, to the flexible coupler 10 at splice region S3 (splice area S3; simple splice S3) inside the lumen of repair suture 10 (braid 10) on the left end 12a and adjacent tail merge splice S1. In some implementations, the distance between the simple splice S3 of the shuttle/pull device 40 and the tail merge splice S1 may be less than about 1 mm. The shuttle/pull device 40 may be any suture passing instrument or a shuttle link such as a FiberLink™ or a Nitinol loop provided with a loop/eye/eyelet. Shuttle/pull device 40 includes shuttle suture 41 and an eyelet/loop 43 for passing the flexible coupler 10.

As detailed below, thin tapered end 13a and shuttle suture 41 are brought together to pass through splice S4 (FIG. 3) within the end 12a of braid 10 and to form two loops 50a, 50b (FIG. 4). Loop 50a is a tensionable, adjustable loop. Loop 50a may be a flexible, closed, knotless, tensionable, continuous loop having an adjustable perimeter and adjustable length. Loop 50b is a static loop. Loop 50b may be a flexible, closed, knotless, continuous loop having a fixed perimeter and length.

Reference is now made to FIGS. 3 and 4. To form loops 50a, 50b an exemplary shuttling device such as a needle (not shown) may be passed within tapered end 12a from D1 to D2 at splice S4. The needle then pierces tapered end 12a at pierce point D3 (FIG. 3). Thin tapered end 13a of flexible coupler 10 together with shuttle suture 41 (the non-looped end of the shuttle/pull device 40) are then threaded onto the needle and then both passed and spliced through the left end 12a at splice S4 (splice region S4; splice area S4) in the left end 12a (FIG. 3). When the needle is pulled out, tapered end 13a and shuttle suture 41 form two loops: an adjustable loop 50a adjacent a static loop 50b, and as part of knotless mechanism 199 (FIG. 4). Once inserted into bone, loop 50a may be reduced (e.g., the perimeter and length of loop 50a may be reduced) by pulling on the exposed suture limb 13a to allow the loop 50a to collapse, while the perimeter and length of loop 50b remain constant.

In some implementations, splice S4 may be spaced apart from splice S3 by a distance D. In some implementations, distance D may be of about 8 mm to about 12 mm, preferably about 10 mm. FIG. 4 illustrates surgical construct 100 in the assembled form and as provided to the surgeon (pre-assembled and ready for insertion). Surgical construct 100 may be inserted into bone via a self-punching driver or, alternatively, by inserting flexible coupler 10 with two loops 50a, 50b of surgical construct 100 into a drilled hole in bone 90.

Surgical assembly 100 with shuttle/pull device 40 may be released from the driver, and the driver removed. Pulling on the exposed suture limb 13a allows loop 50a to reduce its length and perimeter and to collapse within the bone hole.

Free end 13a of flexible coupler 10 may be subsequently passed around or through tissue 80 and then through the eyelet/loop 43 of suture passing device 40. Suture passing device 40 may then be pulled, thereby pulling free end 13a of the flexible coupler 10 towards the body of the fixation device, inside of the braid 10 (at splice S3 (splice region S3) in FIGS. 2 and 3) and then exiting the braid 10 to form another loop 50c. Loop 50c may be a knotless loop. Loop 50c may be a flexible, closed, continuous. Loop 50c may be a tensionable loop with an adjustable perimeter and length. Free end 13a forms a splice inside the braid 10. The suture end 13a of flexible coupler 10 may then be tensioned and cut. Alternatively, suture end 13a may be used for other tissue repairs that could include knotted repairs, such as lateral tissue compression with accompanying interference anchors, for example.

FIG. 5 illustrates a schematic tissue repair 101 (e.g., tendon or ligament repair) with exemplary surgical construct 100 of FIG. 4, to secure a first tissue 80 (for example, soft tissue such as tendon 80) to a second tissue 90 (for example, bone 90). Once surgical coupler 10 (braid 10) of surgical construct 100 has been inserted and secured within a hole 92 in bone 90 and knotless, tensionable loop 50a has been reduced (collapsed), the flexible end 13a of flexible coupler 10 may be passed around or through tissue 80 and then through eyelet 43 of shuttle/pull device 40. The shuttle/pull device 40 may then be pulled out of the braid 10 and out of the surgical site, to allow the flexible coupler 13a to pass through the braid 10 (passing through itself at splice S3 within the body of braid 10) to form loop 50c around tissue 80. Loop 50c may be self-locking and cinching loop. Loop 50c may be flexible, continuous, closed loop. Loop 50c may be a knotless loop with an adjustable perimeter and length.

Free end 13a of the flexible coupler 10 may be pulled to shrink the construct and loop 50c, and to compress the tendon to bone, providing a final repair/construct 101 with increased compression of tissue.

The construct may secure tissue (arthroscopic or endoscopic) with a suture that does not require the surgeon to tie a knot. The construct pulls soft tissue together. The construct may include a flexible coupler that is not tapered. The construct may include a flexible coupler that is tapered/furcated. The furcation(s) in the suture allow the construct to improve tissue cut-through resistance (by reducing the “cheese wire” effect) which is important in many clinical applications. The knotless surgical construct may be employed in knotless fixation of first tissue to second tissue, for example, fixation of soft tissue to bone.

In some implementations, ends 12, 12a, 13, 13a of flexible coupler 10 may be tapered ends that may have similar or different diameters. In some implementations, the diameters of ends 13, 13a may be smaller than the diameters of ends 12, 12a. In some implementations, flexible coupler 10 may be a single strand suture. In some implementations, flexible coupler 10 may be a single braided suture.

The constructs, systems, and assemblies of the present disclosure may be employed in numerous knotless soft tissue repairs and fixations, for example, fixation of soft tissue to bone.

A surgical construct 100 may comprise a flexible coupler 10 (suture or braid 10) with a tapered area 12a, 13a on each side/end of the flexible coupler 10; and a shuttle/pull device 40 attached to the flexible coupler 10. Tapered areas 12a, 13a may have similar or different diameters/widths. Tapered areas 12a, 13a may have diameters smaller than the diameter of flexible coupler 10. The surgical construct 100 may be a soft anchor. The flexible coupler 10 may be a braid, round suture, round-flat-round suture, or suture tape. The ends 12, 12a, 13, 13a may form at least one splice within the flexible coupler 10; a static loop 50b adjacent an adjustable loop 50a; and at least one loop 50c around a second tissue 80 to be secured to a first tissue 90. Loop 50c may be an adjustable, knotless, closed, continuous loop.

A soft anchor 100 may include a braid 10 terminating into a smaller diameter suture 12a, 13a. The braid 10 may act as an anchoring body when secured into a first tissue 90. The smaller diameter suture 12a, 13a may form at least one splice S1, S2, S3, S4 within the braid 10 and at least one loop 50c around a second tissue 80 to be secured to the first tissue 90, and as part of a knotless, self-locking mechanism 199 and repair 101.

Methods of soft tissue repair 101 which do not require tying of knots and allow adjustment of both the tension of the suture and the location of the tissue with respect to the bone, while providing a self-locking mechanism, are disclosed.

A method of knotless tissue repair 101 may comprise inter alia the steps of: (i) securing a braid 10 of a surgical construct 100 into a first tissue 90, the braid 10 being preloaded with a shuttle/pull device 40 and having at least one tapered end 12a, 13a; and (ii) passing one of the at least one tapered ends 12a, 13a around or through a second tissue 80 to be positioned relative to the first tissue 90 and then through the braid 10 by employing the shuttle/pull device 40, to form loop 50c around the second tissue 80. The shuttle/pull device 40 may be attached to a first loop 50a adjacent a second loop 50b formed with one of the ends 12a, 13a. The first loop 50a may be a tensionable, adjustable loop. The second loop 50b may be a static, fixed loop. Loop 50c may be a knotless, closed, adjustable, self-locking, flexible, continuous loop. Loop 50c may have an adjustable perimeter.

As detailed above, when the braid 10 is inserted, the braid 10 has at least one repair suture limb 12a, 13a and also at least one shuttle link 41 (shuttle suture 41). The braid resides within the bone and acts as a soft anchor (as a body of a soft anchor). The repair suture limb(s) resides on top of the bone. The repair suture may be passed around or through the tissue, and then shuttled through the braid and spliced within the braid. The steps may be repeated if additional links are present.

Braid 10 and ends 12, 12a, 13, 13a may include any flexible material, for example, multifilament, braided, knitted, woven suture, or including fibers of ultrahigh molecular weight polyethylene (UHMWPE) or the FiberWire® suture (disclosed in US 6,716,234, the disclosure of which is hereby incorporated by reference in its entirety herein). FiberWire® suture is formed of an advanced, high-strength fiber material, namely ultrahigh molecular weight polyethylene (UHMWPE), sold under the tradenames Spectra (Honeywell) and Dyneema (DSM) fibers, braided with at least one other fiber, natural or synthetic, to form lengths of suture material.

Braid 10 and ends 12, 12a, 13, 13a may be also formed of a stiff material, or combination of stiff and flexible materials, particularly for the regions of the coupler that are passed/spliced through the body of the coupler and depending on whether they are employed with additional fixation devices. Braid 10 and ends 12, 12a, 13, 13a may be also coated and/or provided in different colors for easy manipulation during the surgical procedure. The knotless constructs and self-locking soft anchors of the present disclosure may be used with any type of flexible material or suture that may be weaved or passed through itself.

Various structural elements of surgical construct 100 may be visually coded, making identification and handling of the sheath and suture legs simpler. Easy identification of suture in situ is advantageous in surgical procedures, particularly during arthroscopic surgeries, endoscopic and laparoscopic procedures.

The surgical constructs of the present disclosure may be employed in endoscopic surgery. The term “endoscopic surgery” refers to surgical procedures within a patient's body through small openings as opposed to conventional open surgery through large incisions. Additionally, surgical constructs as disclosed herein may be utilized in other general surgical and specialty procedures such as soft tissue repairs.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed:

1. A soft anchor, comprising:
a suture with two ends;
a static loop and an adjustable loop formed with one of the two ends; and
a shuttle/pull device attached to one of the two ends.

2. The soft anchor of claim 1, wherein the adjustable loop has an adjustable perimeter, and wherein the static loop has a fixed perimeter.

3. The soft anchor of claim 1, wherein the adjustable loop is adjacent the static loop.

4. The soft anchor of claim 1, wherein the suture resides in bone and wherein one of the two ends forms at least another loop around soft tissue to be attached to bone.

5. The soft anchor of claim 4, wherein the at least another loop is a knotless, continuous, adjustable, flexible, self-locking loop.

6. The soft anchor of claim 1, wherein at least one of the two ends is a tapered end.

7. The soft anchor of claim 6, wherein the two ends are tapered ends.

8. The soft anchor of claim 7, wherein one of the two tapered ends is formed by bifurcating a first end of the suture to form two bifurcated limbs, and passing one of the two bifurcated limbs through the other of the two bifurcated limbs to form a splice within the other of the two bifurcated limbs.

9. The soft anchor of claim 8, wherein the other of the two tapered ends is formed by bifurcating twice a second end of the suture to form two bifurcated limbs and two quadfurcated limbs, and passing one of the two bifurcated limbs through the other of the two bifurcated limbs to form a splice within the other of the two bifurcated limbs, and passing one of the two quadfurcated limbs through the other of the two quadfurcated limbs to form a splice within the other of the two quadfurcated limbs.

10. The soft anchor of claim 7, wherein the suture and the two tapered ends are formed as a one-piece construct.

11. The soft anchor of claim 10, wherein the suture and the two tapered ends are formed as a one-piece machine taper construct.

12. The soft anchor of claim 7, wherein one of the two tapered ends forms at least one splice within the other of the two tapered ends.

13. The soft anchor of claim 1, wherein the suture is elastic.

14. A surgical construct comprising:
a suture having a first tapered end and a second tapered end, wherein each of the first tapered end and the second tapered end has a diameter smaller than a diameter of the suture; and
a shuttle/pull device attached to one of the first and second tapered ends.

15. The surgical construct of claim 14, wherein one of the first and second tapered ends forms two loops by being passed through the other of the first and second tapered ends in a direction about parallel to a longitudinal axis of the suture.

16. The surgical construct of claim 15, wherein one of the two loops is a tensionable loop with an adjustable perimeter, and wherein other of the two loops is a fixed loop with a fixed perimeter.

17. The surgical construct of claim 15, wherein the suture with the two loops are configured to reside within bone and to act as an anchoring body.

18. The surgical construct of claim 17, wherein another of the first and second tapered ends is configured to reside on top of the bone and to attach soft tissue to the bone.

19. The surgical construct of claim 18, wherein the another of the first and second tapered ends is configured to pass through an eyelet of the shuttle/pull device and to form a third loop around the soft tissue to be attached to the bone.

20. A method of knotless tissue repair comprising:
securing, in a first tissue, a soft anchor consisting of a braid terminating into a first end and a second end, and a shuttle/pull device attached to one of the first and second ends, wherein the first end is opposite the second end, and wherein the first end forms an adjustable loop adjacent a static loop; and
forming at least another loop around a second tissue to be attached to the first tissue by passing the second end around or through the second tissue and then through the braid and by employing the shuttle/pull device.

21. The method of 20, further comprising:
passing the first end through a body of the braid to form a splice and the adjustable loop adjacent the static loop;
inserting the soft anchor into bone;
reducing the adjustable loop;
subsequently, passing the second end around or through the second tissue; and
passing the second end through the body of the braid by employing the shuttle/pull device, to form a splice and the least another loop around the second tissue.

22. The method of claim 20, wherein the first tissue is bone and the second tissue is tendon or ligament.

23. The method of claim 20, wherein the soft anchor is an all-suture anchor formed of a single suture material.

24. The method of claim 20, wherein the adjustable loop is a knotless, closed, tensionable, continuous loop, and wherein the static loop is a knotless, closed, continuous, non-adjustable loop.

25. The method of claim 20, wherein the at least another loop is a knotless, closed, continuous, adjustable, tensionable loop.

26. The method of claim 20, wherein at least one of the first end and the second end is tapered.

27. The method of claim 20, wherein both the first end and the second end are tapered.
